(19) United States
(12) Patent Application Publication
(10) Pub. No.: US 2024/0315697 A1
(43) Pub. Date: Sep. 26, 2024

(54) DEVICES, SYSTEMS, AND METHODS FOR HOLDING ANATOMICAL STRUCTURES IN APPOSITION

(71) Applicant: BOSTON SCIENTIFIC SCIMED, INC., MAPLE GROVE, MN (US)

(72) Inventors: John O'Driscoll, Galway (IE); Fionn Stapleton, Galway (IE); David Collins, Galway (IE); Darren Gerard Curran, Galway (IE); Ryan Desmond Lynch, Roscommon (IE); Jack Mitchell, Galway (IE)

(73) Assignee: BOSTON SCIENTIFIC SCIMED, INC., MAPLE GROVE, MN (US)

(21) Appl. No.: 18/609,322

(22) Filed: Mar. 19, 2024

Related U.S. Application Data
(60) Provisional application No. 63/453,227, filed on Mar. 20, 2023.

(51) Int. Cl. A61B 17/11

(52) U.S. Cl. CPC A61B 17/1114; A61B 2017/1139

(57) ABSTRACT

An implantable medical device, such as a stent, having a dynamic feature allowing movement of one or both of the ends of the implantable medical device relative to the intermediate section therebetween. The implantable medical device may be sized, shaped, configured, and/or dimensioned to hold anatomical structures in apposition. The dynamic section allows relative movement of the apposed anatomical structures without causing migration of the ends of the implantable medical device which are anchored with respect to the anatomical structures.

### CROSS-REFERENCE TO RELATED APPLICATIONS

This application claims the benefit of priority under 35 U.S.C. § 119 to U.S. Provisional Application No. 63/453,227, filed Mar. 20, 2023, the entire disclosure of which is hereby incorporated by reference herein for all purposes.

### FIELD

The present disclosure relates generally to the field of implantable medical devices, systems, and methods. More particularly, the present disclosure relates to medical devices, systems, and methods for holding anatomical structures in apposition.

### BACKGROUND

Various devices such as stents are extended across anatomical structures for various purposes. For instance, various stents are known for establishing connections between anatomical structures. Some such connections are made simply to hold tissue in apposition, whereas some such connections also establish fluid communication between anatomical structures such as organs, cavities, lumens, passages, etc. In some instances, it is desirable to create a semi-permanent or permanent anastomosis allowing fluid flow or drainage from one anatomical structure to another anatomical structure. For example, in various gastrointestinal (GI) procedures (e.g., gastric bypass surgery), lumen apposing stents may be used to form an anastomosis in the GI system, such as a gastrojejunostomy between the stomach and the jejunum. The gastrojejunostomy facilitates flow of food particulate, liquid, chyme, etc., from the stomach to the lower GI tract, bypassing the pylorus and the duodenum (e.g., approximately the first 1.5 m of the small intestine, where most food, fats, and nutrients are digested). Such procedure is considered to be less invasive than prior Roux-en-Y surgical bypass procedures, and may be reversible. As may be appreciated, it is desirable for the anastomosis device to remain securely in place until removal is desired or medically indicated. In general, and more broadly, in various procedures or uses of an implantable medical device extending across anatomical structures, it may be desirable for the device to remain in place for a prolonged period of time (e.g., days, weeks, months, even upwards of six to twelve months). However, when positioned across two different anatomical structures which may move with respect to each other, an implantable medical device may become dislodged by the movement of the anatomical structures against which the stent is positioned and anchored, and may migrate from the deployment site. Accordingly, there remains an ongoing need for improved anti-migration features and/or configurations of implantable devices for extending across apposed, generally non-adherent, anatomical structures, and improvements to associated systems and methods as well.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an implantable medical device is configured to hold first and second anatomical structures in apposition between a first end and a second end of the implantable medical device. The implantable medical device has an intermediate section defined between the first end and the second end of the implantable medical device and extending through the anatomical structures. In some aspects, the first end of the implantable medical device is configured to be anchored with respect to the first anatomical structure. In some aspects, the second end of the implantable medical device is configured to be anchored with respect to the second anatomical structure. In some aspects, the intermediate section is sized, shaped, configured, and/or dimensioned to hold the anatomical structures in apposition with the first end of the implantable medical device anchored with respect to the first anatomical structure and the second end of the implantable medical device anchored with respect to the second anatomical structure. In some aspects, the intermediate section includes a dynamic section configured to allow movement of the first end of the implantable medical device relative to the second end of the implantable medical device.

The dynamic section is optionally substantially coextensive with the intermediate section.

In some embodiments, at least the first end and the second end of the implantable medical device are formed of interconnected filaments; and the dynamic section of the intermediate section has a structure different from the interconnected filament structure of the intermediate section.

The dynamic section optionally is formed of a substantially solid polymeric wall.

In some embodiments, the intermediate section includes a section formed of interconnected filaments; and the dynamic section includes a first dynamic section coupling a first end of the interconnected filament section of the intermediate section to the first end of the implantable medical device, and a second dynamic section coupling a second end of the interconnected filament section of the intermediate section to the second end of the implantable medical device.

In some embodiments, a first retention member extends radially-outwardly along the first end of the implantable medical device; a second retention member extends radially-outwardly along the second end of the implantable medical device; the first retention member and the second retention member are formed from filaments interconnected in a first pattern; and at least a portion of the intermediate section is formed from filaments interconnected in a second pattern different from the first pattern. In some embodiments, the second pattern imparts a different degree of flexibility to the intermediate section than the flexibility imparted by the first pattern to the first retention member and to the second retention member. In some embodiments, the dynamic section includes a first dynamic section coupling a first end of the portion of the intermediate section formed from interconnected filaments with the first retention member, and a second dynamic section coupling a second end of the portion of the intermediate section formed from interconnected filaments with the second retention member.

In some aspects, a lumen is defined through the implantable medical device to form an anastomosis between the apposed anatomical structures. Optionally, the dynamic section is reinforced to maintain the lumen through the implantable medical device in an open configuration.

Optionally, the intermediate section includes more than one dynamic section.

In some aspects, the implantable medical device is shiftable from a delivery configuration to an expanded deployed configuration. In some aspects, the first end of the implantable medical device expands when the implantable medical device shifts to the deployed configuration to define a radially-outwardly extending first retention member. Additionally or alternatively, the second end of the implantable medical device expands when the implantable medical device shifts to the deployed configuration to define a radially-outwardly extending second retention member. Additionally or alternatively, a first end of the intermediate section is coupled to the first retention member. Additionally or alternatively, a second end of the intermediate section is coupled to the second retention member.

In some aspects, the intermediate section is sized, shaped, configured, and/or dimensioned to hold adjacent non-adherent anatomical structures in apposition.

In accordance with various principles of the present disclosure, an implantable medical device is configured to hold first and second anatomical structures in apposition, the implantable medical device having a first end and a second end with an intermediate section extending therebetween. The implantable medical device has a first retention member sized, shaped, configured, and/or dimensioned to anchor a first end of the implantable medical device with respect to the first anatomical structure; a second retention member sized, shaped, configured, and/or dimensioned to anchor a second end of the implantable medical device with respect to the first anatomical structure; and a dynamic section defined along the intermediate section to allow the intermediate section to be movable with respect to the first retention member and the second retention member.

In some embodiments, the first retention member and the second retention member are formed from interconnected filaments; and the dynamic section has a structure different from the interconnected filament structure of the first retention member and the second retention member.

In accordance with various principles of the present disclosure, a method of holding a first anatomical structure of a patient in apposition with a second anatomical structure of the patient includes anchoring a first end of an implantable medical device with respect to the first anatomical structure; extending an intermediate section of the implantable medical device across the first anatomical structure and the second anatomical structure; anchoring a second end of the implantable medical device with respect to the second anatomical structure; and allowing a dynamic section of the implantable medical device to flex with respect to at least one of the retention members. In some aspects, the intermediate section is sized, shaped, configured, and/or dimensioned to hold the first and second anatomical structures in apposition with the first end of the implantable medical device anchored with respect to the first anatomical structure and the second end of the implantable medical device anchored with respect to the second anatomical structure.

Optionally, a lumen is defined through the implantable medical device, and the method further includes forming an anastomosis between the first and second anatomical structures. In some aspects, the first anatomical structure is the jejunum of the patient and the second anatomical structure is the stomach of the patient, the method further including forming a gastrojejunostomy.

In some aspects, the method further includes delivering the implantable medical device, in a delivery configuration, transluminally through the patient; deploying the first end of the implantable medical device in the first anatomical structure and allowing the first end of the implantable medical device to expand to form a first retention member anchored against a distal side of the first anatomical structure; extending the intermediate section through a wall of the first anatomical structure and a wall of a second anatomical structure; and deploying the second end of the implantable medical device in the second anatomical structure and allowing the second end of the implantable medical device to expand to form a second retention member anchored against a proximal side of the second anatomical structure.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an implantable medical device formed in accordance with various aspects of the present disclosure and positioned in a schematic representation of a gastrointestinal environment.

FIG. 2 illustrates an elevational view of an example of an embodiment of an implantable medical device formed in accordance with various principles of the present disclosure and illustrated extending across two different anatomical walls.

FIG. 3 illustrates another example of an embodiment of an implantable medical device formed in accordance with various principles of the present disclosure.

FIG. 4 illustrates another example of an embodiment of an implantable medical device formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Implantable medical devices formed in accordance with various principles of the present disclosure are configured to extend across adjacent or apposed anatomical structures and to be held in place with respect thereto. In accordance with various further principles of the present disclosure, such implantable medical devices are formed to hold the anatomical structures in apposition with respect to each other. Implantable medical devices formed in accordance with various principles of the present disclosure may also be formed to establish a flow or access passage (e.g., anastomosis) between the apposed anatomical structures. The implantable medical devices are configured and deployed to be anchored or otherwise held in place with respect to the deployment site, such as with respect to tissue walls at the deployment site. For instance, implantable medical devices formed in accordance with various principles of the present disclosure are configured with one or more retention or anchor structures (which may alternatively be referenced as retention members or flanges). Additionally or alternatively, implantable medical devices formed in accordance with various principles of the present disclosure are designed to encourage tissue ingrowth therewith to improve migration resistance. It will be appreciated that terms such as hold or anchor (and other grammatical forms thereof) may be used interchangeably herein with terms (and grammatical forms thereof) such as, without limitation, affix, attach, associate, couple, engage, embed, implant, retain, purchase, secure, etc., without intent to limit. Additionally, it is noted that reference may be made alternately to an anatomical site, deployment site, treatment site, delivery site, etc., without intent to limit.

One challenge of deploying an implantable medical device across anatomical structures is that the anatomical structures (particularly if non-adherent) may be movable with respect to each other, and may remain at least somewhat movable with respect to each other even after an implantable medical device is deployed across such anatomical structures to hold them in apposition. Relative movement of the anatomical structures may cause movement of the implantable medical device, may interfere with implantation of the implantable medical device (e.g., may interfere with tissue ingrowth), and may cause dislodging/migration of the implantable medical device from the deployment site. For instance, an implantable medical device deployed across a stomach and a portion of the intestines (e.g., jejunum), such as to form a gastrojejunostomy (“GJ”), is subjected to various forces tending to cause migration of the implantable medical device. Because intestines are typically motile, particularly relative to the stomach, the intestines may move laterally with respect to, as well as away from, the stomach. Additionally or alternatively, the weight of the intestines and gravity may cause the intestines to be pulled away from the stomach, exerting a pull out force on the implantable medical device. Accordingly, it is desirable to improve retention of an implantable medical device with respect to its deployment site (e.g., with respect to apposed anatomical structures across which the implantable medical device is deployed). As noted above, various implantable medical devices may be designed to encourage tissue ingrowth therewith to improve migration resistance with respect to the deployment site. However, such tissue ingrowth does not happen immediately. Therefore, it is desirable to improve retention of such implantable medical devices during a dwell time at least sufficient to establish tissue ingrowth with respect to the deployment site.

Examples of embodiments of implantable medical device disclosed herein have an elongate body with ends configured to anchor the implantable medical device with respect to an anatomical structure thereof, and an intermediate section extending between the ends. In some embodiments, such as those described herein, retention members (which may alternatively be referenced as flanges, anchoring sections, etc., without intent to limit) are defined/provided at the ends of the implantable medical device, and are configured to maintain the implantable medical device in place with respect to anatomical structures to be held in apposition by the implantable medical device. The intermediate section (which may alternatively be referenced as a saddle region) extends between the retention members and through the anatomical structures across which the implantable medical device is deployed. The retention members may have diameters which are larger than the diameter of the intermediate section, and may additionally be otherwise configured to retain the implantable medical device in place with respect to the deployment site.

In accordance with various principles of the present disclosure, at least a portion of the intermediate section includes one or more dynamic sections (which may alternatively be referenced as a portion, region, etc.) configured to allow transverse and/or radial forces to move or otherwise impact the intermediate section without affecting the retention members. As such, transverse and/or radial forces do not get transmitted to the retention members. The apposed anatomical structures with respect to which the retention members are anchored, as well as the retention members, may thus move and flex independently of each other (without forces impacting the intermediate section affecting the secured positions of the retention members with respect to the apposed anatomical structures) and thereby allow for the formation of the anastomosis therebetween. For instance, the dynamic section of the intermediate section of the implantable medical device may continuously flex and move as the apposed anatomical structures move with respect to each other, thereby reducing the forces acting on an individual retention member, and therefore reducing migration rates of the implantable medical device. It will be appreciated that references to a dynamic section in the singular may be considered to include more than one dynamic section. In some embodiments, the dynamic section is substantially coextensive with (e.g., defined along substantially the entirety of) the intermediate section. In some embodiments, the intermediate section may be considered to itself be the dynamic section.

In accordance with various principles of the present disclosure, the dynamic section allows movement, flexing, bending, contraction, etc., of the dynamic section relative to other sections of the implantable medical device upon relative movement of the apposed anatomical structures across which the implantable medical device has been deployed. The dynamic section may be formed and/or configured to be distinct from other sections of the implantable medical device. In some embodiments, the dynamic section has a structure and/or material properties different from the structure and/or material properties of the retention members. For instance, the dynamic section may be formed of a material or combination of materials different from the material or combination of materials forming the retention members and/or other sections of the intermediate section, such as distal or proximal to the dynamic section. Additionally or alternatively, the dynamic section may have a structure different from, or may be otherwise formed differently from, the retention members and/or other sections of the intermediate section, such as distal or proximal to the dynamic section. In some embodiments, at least a portion of the intermediate section of an implantable medical device formed in accordance with various principles of the present disclosure is coupled with (e.g., an extension of) the retention members. In such embodiments, ends of a dynamic portion of an intermediate section are coupled with the portion of the retention members (e.g., extended from and optionally also a part of) the retention members.

In accordance with various further principles of the present disclosure, an implantable medical device is configured to form an anastomosis between/across anatomical structures with respect to which the implantable medical device has been deployed. In accordance with various principles of the present disclosure, an implantable medical device may be configured not only to prevent migration thereof during formation of an anastomosis, but also to maintain the required integrity of the implantable medical device to prevent leakage through/from the anastomosis (e.g., transversely through, rather than longitudinally and out the ends of the device). For example, an implantable medical device formed in accordance with various principles of the present disclosure may be configured and deployed to form an anastomosis between the stomach and jejunum of a patient, and to prevent leakage through the device and into the peritoneum during the formation of the anastomosis. For instance, a coating or liner or other impermeable or semi-impermeable material may extend along (and optionally be coupled to, such as coating or otherwise) the implantable medical device. More particularly, the coating or liner may be extended over or within, attached to, formed with, or otherwise associated with a wall forming the implantable medical device. The coating may be evenly applied along the entire length of the implantable medical device, or may be unevenly applied, for example, increasing in thickness proximally or distally (e.g., forming ridges), or covering only part of the implantable medical device (e.g., forming openings, slits, slots, etc.).

An example of an embodiment of an implantable medical device formed in accordance with various principles of the present disclosure has a separately formed dynamic section along an intermediate section between retention members of the implantable medical device. Various characteristics or properties of the dynamic section allow anatomical structures held in apposition by the implantable medical device to move independently of each other, while allowing the retention members of the implantable medical device to remain in place with respect to the anatomical structures. For instance, the dynamic section may be a flexible section (e.g., a flexible cylinder/tube) which absorbs or moves with forces on the intermediate section of the implantable medical device so as not to disturb the retention members of the implantable medical device. The dynamic section may thus facilitate anchoring of the implantable medical device, the formation of an anastomosis, and/or maintenance of a lumen extending longitudinally through the implantable medical device in an open configuration to allow the passage of materials therethrough.

Various embodiments of implantable medical devices, systems, and associated methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the accompanying drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

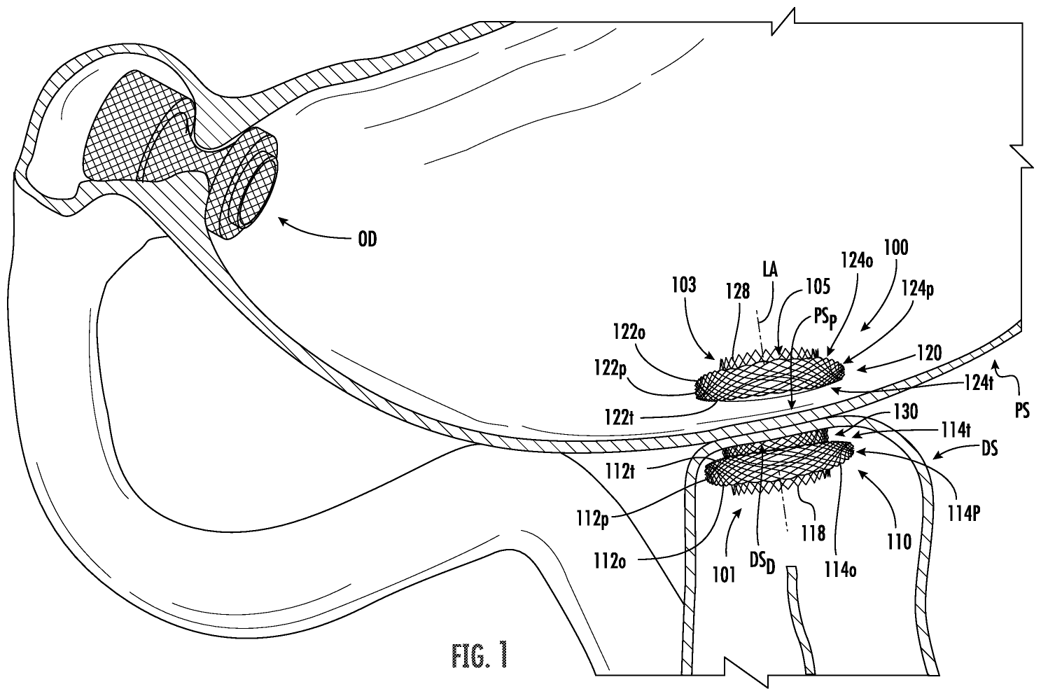

Turning now to the drawings, an example of an embodiment of an implantable medical device 100 formed in accordance with various principles of the present disclosure is illustrated in FIG. 1 as holding a proximal anatomical structure PS and a distal anatomical structure DS in apposition with respect to each other. More particularly, the implantable medical device 100 is illustrated in a gastrointestinal system of a patient forming an anastomosis (e.g., gastrojejunostomy) between the stomach (as the proximal anatomical structure PS), and a portion of the small intestines (as the distal anatomical structure DS), such as the jejunum. An additional device, such as an occlusive device OD, may be deployed with respect to the pylorus to occlude the pylorus so that materials from the stomach are directed to the gastrojejunostomy rather than through the pylorus. It will be appreciated that implantable medical devices formed in accordance with various principles of the present disclosure may be beneficial in procedures other than metabolic procedures, such as with any other procedure or application where migration of an implantable medical device is a concern. References to gastrojejunostomies are solely for the sake of example, without intent to limit.

The example of an embodiment of an implantable medical device 100 illustrated in FIG. 1 has a distal end 101 extended into and anchored with respect to the distal anatomical structure DS, and a proximal end 103 extended into and anchored with respect to the proximal anatomical structure PS. The implantable medical device 100 may be delivered in any of a variety of manners, such as those known to those of ordinary skill in the art, typically delivering the distal end 101 first, and then deploying the proximal end 103. For instance, the implantable medical device 100 may be delivered in a compact (e.g., constricted, collapsed, etc.) delivery configuration capable of fitting through an elongate tubular element (e.g., endoscope, catheter, sheath, etc.) configured for transluminal/intraluminal delivery through a patient (e.g., transcatheter, in contrast with an open surgical procedure) to a deployment site. For instance, the implantable medical device may be delivered via a natural orifice transluminal endoscopic surgery (NOTES) procedure, considered to be simpler and less invasive than open surgical procedures (such as for Roux-en-Y procedures). The implantable medical device 100 may be advanced distally out of the elongate tubular element, and/or the elongate tubular element may be proximally withdrawn, to deploy the implantable medical device 100 at the deployment site. Once deployed, the implantable medical device 100 may expand to a deployed configuration generally corresponding to the deployment site (e.g., to engage and be securely implanted with respect to the deployment site). The implantable medical device 100 may be self-expanding (i.e., capable of expanding on its own from the delivery configuration into the deployed configuration, such as by being formed, at least in part, by a shape memory material), or may expand with the assistance of another device, such as a balloon. The present disclosure is not to be limited by the manners of delivering, or the delivery devices or systems used to deliver, the implantable medical device 100.

The illustrated implantable medical device 100 is sized, shaped, configured, and/or dimensioned to hold the distal anatomical structure DS and the proximal anatomical structure PS in apposition with respect to each other. In accordance with various principles of the present disclosure, in embodiments in which the implantable medical device 100 is to hold anatomical structures in apposition, the length of the implantable medical device 100, between the distal end 101 and the proximal end 103 is determined based on the nature and various other properties of the anatomical structures to be held in apposition by the implantable medical device 100. For instance, the length of the implantable medical device 100 may be determined based on the thickness of the tissue walls of the anatomical structures to be held in apposition, how close the anatomical structures are to be held with respect to each other, etc. It will be appreciated that other properties of the implantable medical device 100 may also be selected based on the anatomical structures across which the implantable medical device 100 is to be deployed.

As used to hold anatomical structures in apposition, the example of an embodiment of an implantable medical device 100 illustrated in FIG. 1 includes a distal retention member 110 along the distal end 101 of the implantable medical device 100, and a proximal retention member 120 along a proximal end 103 of the implantable medical device 100, with an intermediate section 130 of the implantable medical device 100 defined therebetween. The retention members 110, 120 are sized, shaped, configured, and/or dimensioned to retain the implantable medical device 100 with respect to the deployment site. More particularly, the size, shape, configuration, and/or dimensions of the retention members 110, 120 may be selected to seat against the distal anatomical structure DS and the proximal anatomical structures PS, respectively, such as against anatomical walls of the anatomical structures DS and PS (e.g., tissue walls extending radially outwardly from the body passage through which the intermediate section 130 of the implantable medical device 100 extends). As such, the retention members 110, 120 typically are transverse to the intermediate section 130 of the implantable medical device (e.g., along a longitudinal axis LA along which the implantable medical device 100, or at least a majority of the length thereof, generally extends). Typically, the retention members 110, 120 are wider (in a radial direction transverse to the longitudinal axis of the body passage) than the intermediate section 130 to resist migration of the implantable medical device 100 with respect to the deployment site by holding the retention members 110, 120 against any pull out forces exerted on the implantable medical device 100.

The retention members 110, 120 of an embodiment of an implantable medical device 100 illustrated in FIG. 1 are proximate the ends of the intermediate section 130, and are spaced apart from each other to hold anatomical structures DS and PS, such as non-adherent anatomical structures, in apposition. The anatomical structures DS and PS may be adjacent each other, in contrast with spaced apart anatomical structures with another anatomical structure therebetween. The intermediate section 130 is extended through the anatomical structures DS and PS, such as through a body passage defined therethrough. It will be appreciated that reference to a body passage includes naturally-existing passages as well as medically-created passages (e.g., a passage created with the use of a medical instrument, such as a cutting instrument, such as between a stomach and jejunum, as illustrated in FIG. 1) or otherwise. As such, the overall length of the implantable medical device 100 from one end 101 to the other end 103 may be based on the longitudinal extent desirable or necessary to hold the non-adherent anatomical structures DS and PS in apposition, such as to form an anastomosis therebetween (e.g., formed by tissue growth of the apposed anatomical walls with respect to each other as well as with respect to the implantable medical device 100). Although the intermediate section 130 may be seen in the illustration of FIG. 1, it will be appreciated that the intermediate section 130 may be sized to extend through the apposed walls of the anatomical structures DS and PS and not be visible beyond the walls (e.g., beyond the non-apposed sides DS_{D} and PS_{P}, such as illustrated in FIG. 1).

In the example of an embodiment of an implantable medical device 100 illustrated in FIG. 1, the distal retention member 110 has a tissue-engaging side 112t facing a wall of the distal anatomical structure DS, and the proximal retention member 120 has a tissue-engaging side 122t facing a wall of the proximal anatomical structure PS. Even more particularly, the tissue-engaging side 112t of the distal retention member 110 engages a distal-facing side DS_{D} of the wall of the distal anatomical structure DS, and the tissue-engaging side 122t of the proximal retention member 120 engages a proximal facing side PS_{P} of the wall of the proximal anatomical structure PS. The tissue-engaging sides 112t, 122t of the retention members 110, 120, respectively, or at least one or more portions thereof, may be sized, shaped, configured, and/or dimensioned to seat against the anatomical structures DS and PS (and distal-facing side DS_{D} and proximal facing side PS_{P} thereof), respectively. It will be appreciated that the retention members 110, 120 may be single-wall elements, wherein the tissue-engaging sides 112t, 122t are sides of a respective retention member 110, 120. Alternatively, at least one of the retention members 110, 120 may be a double-wall retention member. For instance, in the example of an embodiment illustrated in FIG. 1, the illustrated double-walled distal retention member 110 has a first wall 114t defining the tissue-engaging side 112t, and a second wall 1140 defining the outwardly-facing side 1120 of the distal retention member 110 (facing away from the deployment site and the remainder of the implantable medical device 100). A peripheral wall 114p extends between the first wall 114t and the second wall 1140 of the distal retention member 110, defining a peripheral side 112p of the distal retention member 110 (extending between the tissue-engaging side 112t and the outwardly-facing side 1120 of the distal retention member 110). Similarly, the double-walled proximal retention member 120 illustrated in FIG. 1 has a first wall 124t defining the tissue-engaging side 122t, and a second wall 1240 defining the outwardly-facing side 1220 of the proximal retention member 120 (facing away from the deployment site and the remainder of the implantable medical device 100), with a peripheral wall 124p extending between the first wall 124t and the second wall 1240 and defining a peripheral side 122p of the proximal retention member 120. It will be appreciated that only one of the retention members 110, 120 may be formed as a double-wall retention member and the other may be formed as a single wall retention member.

As may be appreciated, if the anatomical structures across which an implantable medical device is deployed shift or move with respect to each other and/or do not remain parallel to each other, then the walls of the anatomical structures may exert pull-out forces on the retention members of the implantable medical device Accordingly, an intermediate section 130 formed in accordance with various principles of the present disclosure is configured to be movable with relative movements of the anatomical structures DS and PS across which the implantable medical device 100 is deployed. Such movement of the intermediate section 130 inhibits transference of such movements of the anatomical structures DS and PS to the retention members 110, 120, thereby reducing pull out forces or other forces on the retention members 110, 120 which may cause the retention members 110, 120 to migrate from their respective deployment sites. It will be appreciated that the ability of the intermediate section 130 to move with relative movements of the anatomical structures DS and PS may additionally or alternatively be referenced herein as the ability to absorb, mitigate, dissipate, etc., relative movements of the anatomical structures DS and PS, without intent to limit.

In some aspects, an implantable medical device 100 formed in accordance with various principles of the present disclosure has an intermediate section 130 with one or more characteristics or properties which are distinct from those of the retention members 110, 120. The distinct characteristics or properties of the intermediate section 130 may extend along the entire length of the intermediate section 130, or may be limited to one or more sections/portions of the intermediate section 130. Such distinct characteristics or properties allow relative movements of the anatomical structures across which the implantable medical device 100 is deployed to move the intermediate section 130 without also moving the retention members 110, 120, and thus not causing the retention members 110, 120 to dislodge and/or migrate. For the sake of convenience, and without intent to limit, the one or more portions of the intermediate section 130 having characteristics or properties distinct from those of the retention members 110, 120 may be referenced as the dynamic section of the intermediate section 130. It will be appreciated that reference to a dynamic section may refer to a single section or more than one section of the intermediate section 130. The distinct characteristics or properties may be differences in material properties and/or material compositions and/or one or more structural properties. For instance, the intermediate section 130 may be formed of a more flexible material than the material of the retention members 110, 120. Alternatively, the intermediate section 130 and the retention members 110, 120 may be formed from similar materials which are constructed to have different structural configurations allowing different flexibilities. It will be appreciated that terms such as properties and characteristics (and/or similar terms) may be used interchangeably herein without intent to limit (unless explicitly indicated), and reference to one term is not meant to exclude the other term (unless explicitly indicated). Similarly, terms such as distinct and different may be used interchangeably herein without intent to limit (unless explicitly indicated), and reference to one term is not meant to exclude the other term.

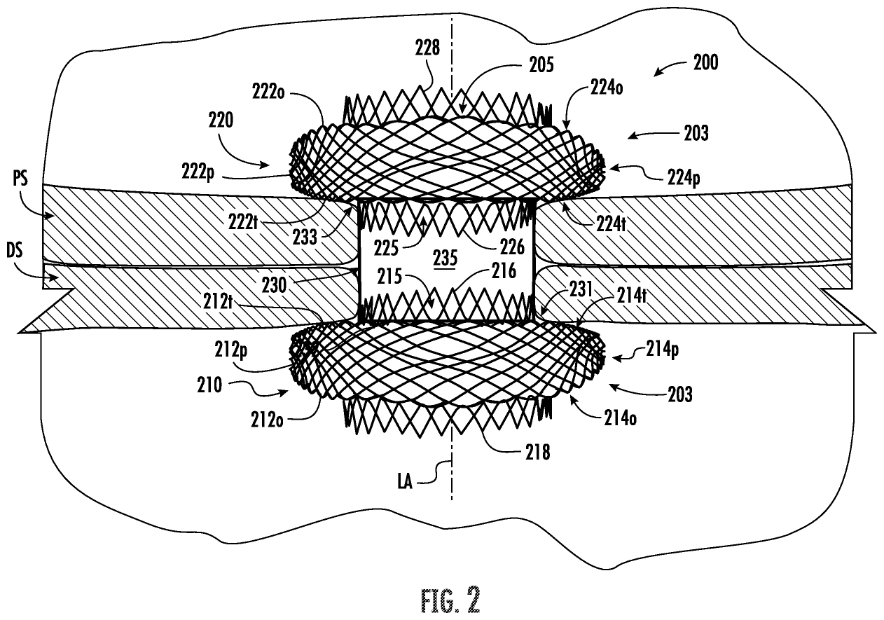

An example of an embodiment of an implantable medical device 200 configured to hold anatomical structures in apposition and having an intermediate section 230 with a dynamic section configured to move with movements of the apposed anatomical structures is illustrated in FIG. 2. The illustrated example of an embodiment has two retention members 210, 220, with an intermediate section 230 therebetween. Although the retention members 210, 220 are illustrated as double-walled retention members with distal tissue-engaging sides 212t, 222t defined by respective first walls 214t, 224t, and with outwardly-facing sides 2120, 2220 defined by second walls 2140, 2240, and with peripheral sides 212p, 222p defined by peripheral walls 214p, 224p (joining the respective first walls 214t, 224t and second walls 2140, 2240), respectively, the distal tissue-engaging sides 212t, 222t and the outwardly-facing sides 2120, 2220 of the retention members 210, 220, respectively, may be defined by opposite sides of single walls of the retention members 210, 220, the present disclosure not being limited in this regard. The intermediate section 230 has a dynamic section with properties/characteristics which are distinct/different from those of the retention members 210, 220 to allow movements of the apposed non-adherent anatomical structures DS and PS to affect the intermediate section 230 without affecting the retention members 210, 220. For instance, the retention members 210, 220 typically are configured to be more resistant to flexing than the dynamic section of the intermediate section 230, such as to retain the implantable medical device 200 in place with respect to the deployment site. In contrast, at least the dynamic section of the intermediate section 230 is typically capable of flexing or otherwise bending or pivoting or deforming so as not to transfer forces to or cause deformation of or otherwise to affect the retention members 210, 220.

In the example of an embodiment illustrated in FIG. 2, the retention members 210, 220 are structurally separate and spaced apart from each other and joined by a distinct intermediate section 230 to allow the retention members 210, 220 and the intermediate section 230 to have properties/characteristics which are distinct/different. The intermediate section 230 may be distinct in any of a variety of manners, such as separately formed from the retention members 210, 220; treated to have different properties than those of the retention members 210, 212; formed with (e.g., using a similar manufacturing process as) the retention members 210, 220 but with a modification thereto (e.g., modification to the material thereof), etc.

The retention members 210, 220 of the example of an embodiment of an implantable medical device 200 illustrated in FIG. 2 may be formed from a plurality of strands which may be interconnected to form a self-supporting structure. It will be appreciated that terms such as strands may be used interchangeably herein with terms such as, without limitation, wires or filaments or ribbons without intent to limit. It will further be appreciated that terms such as, without limitation, interconnected or woven or interwoven or braided or twisted or wrapped or intertwined or knitted or looped (e.g., bobbinet-style) or knotted or counterwound or interengaged or otherwise formed may be used interchangeably herein (optionally in other grammatical forms thereof) without intent to limit. A self-supporting structure may be alternately referenced herein as a stent, framework, scaffold, graft, etc., without intent to limit. The filaments forming the retention members 210, 220 may themselves be formed from a (preferably biocompatible) metal, such stainless steel; and/or an alloy such as a nickel-titanium alloy (e.g., Nitinol), a nickel-tungsten or tungsten alloy, a cobalt-chromium alloy, a cobalt-chromium-nickel based alloy (e.g., Elgiloy®), a nickel-copper alloy, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and subcombinations and other alloys thereof or the like. Additionally or alternatively, the filaments may be formed of a (preferably biocompatible) polymeric material such as polypropylene, polyester, nylon, polyethylene terephthalate (PET), polyetheretherketone (PEEK), poly(methyl methacrylate) (PMMA), polysulfone, polyurethane, polystyrene, polyethylene (PE) (including high-density and low-density PE's), polyethylene naphthalate (PEN), polybutylene terephthalate (PBT), polytrimethylene terephthalate, polyether block amides (PEBA), polyetherimide (PEI), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly paraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, subcombinations, and copolymers thereof, or the like. Additionally or alternatively, the filaments may be formed of a bioabsorbable material, such as polyglycolic acid, lactic acid, poly(lactic-co-glycolic acid), caprolactone, polymers, polydioxanone, cat or bovine intestine, or the like; and/or a natural fiber, such as silk or cotton, or the like. The filaments may be formed of mixtures, composites, or co-constructions of any of the above. Alternatively, the retention members 210, 220 may be formed from a laser-cut tube or bonded elongated elements or another self-supporting structure. In some embodiments, the material from which one or both of the retention members 210, 220 are formed is a shape-memory or heat formable material.

In some embodiments, at least a portion of the walls of an implantable medical device formed in accordance with various principles of the present disclosure have gaps, apertures, openings, interstices, etc., therethrough. Application of a coating material to, over, on, etc. (such terms being used interchangeably herein without intent to limit) at least a portion of an implantable medical device formed in accordance with various principles of the present disclosure may fill in some or all of such gaps, apertures, openings, interstices, etc., to inhibit or prevent flow or leakage of materials therethrough and/or to inhibit tissue ingrowth therein. Additionally or alternatively, a liner may be provided along at least a portion of the implantable medical device. Such coating or liner may be formed of any of a variety of polymeric materials (preferably biocompatible and/or non-degradable, e.g., upon exposure to bodily fluids such as bile), including, for example, silicones, rubbers, polyethylenes, PVDF, urethane, polyurethane, and other thermoplastic elastomers, including mixtures, composites, combinations, subcombinations, copolymers and/or co-constructions thereof.

Whereas the retention members of an implantable medical device such as described above may be configured to resist various lateral and/or axial forces exerted on the implantable medical device and the retention members, the intermediate section extending between the retention members is configured to flex, bend, pivot, or otherwise move with or allow movements of the anatomical structures. Such movement of the intermediate section allows relative movements of the anatomical structures to not be transmitted to the retention members. For instance, pivoting of the walls of the anatomical structures with respect to each other (movement of the planes in which the walls of the anatomical structures lie to be transverse, rather than parallel, to each other) causes pivoting of the intermediate section without causing shifting or other movement of, or transmission of undue forces to, the retention members. As such, forces which may cause migration of the retention members are inhibited or prevented from being transmitted to the retention members.

To be capable of such movement, an intermediate section of an implantable medical device formed in accordance with various principles of the present disclosure includes a dynamic section which may be formed of a flexible material and/or a material formed to have a structure which is more flexible than that of the ends of the implantable medical device (e.g., the retention members). As such, the dynamic section of the intermediate section has a structure and/or material different from that of the ends/retention members. For instance, at least a portion of the intermediate section 230 of the implantable medical device 200 illustrated in FIG. 2 includes a flexible cylinder, tube, sleeve, etc. (such terms, and other grammatical forms thereof, being used interchangeably herein without intent to limit) extending between the retention members 210, 220 of the implantable medical device 200 and defining the dynamic section. The dynamic section of the example of an embodiment illustrated in FIG. 2 may be substantially coextensive with the intermediate section 230 (i.e., substantially the entire length of the intermediate section 230 is formed to function as a dynamic section of the implantable medical device 100). The dynamic section of the implantable medical device 200 has characteristics or properties distinct from those of the retention members 210, 220 in one or more manners.

In the example of an embodiment of an implantable medical device 200 illustrated in FIG. 2, the wall of the intermediate section 230 may be formed from a material more flexible than the material of the retention members 210, 220. For instance, the intermediate section 230 may be formed of a flexible material such as silicone, or other generally flexible and preferably biocompatible material, such as a flexible polymeric material, including, without limitation, polyethylene (PE), polyethylene terephthalate (PET), polypropylene (PP), nylons, polytetrafluorethylene (PTFE), ethylene tetrafluoroethylene (ETFE), fluorinated ethylene propylene (FEP), polyurethane, polyvinyl chloride (PVC), polyetheretherketone (PEEK), polyether-ester, block copolymers such as polyamide and polyether block copolymers (e.g., PEBAX® elastomers), perfluoroalkoxy plastics (PFA), polyvinylidene fluoride (PVDF), thermoplastics and/or thermosets, etc., and composites and/or copolymers thereof. Such a flexible material may be used to form a wall of the implantable medical device 200 (e.g., at least a portion of the intermediate section 230) having the desired flexibility, in contrast with a structure configured to impart structural stability, such as the above-described stent structure of the retention members 210, 220. In some embodiments, as described above, the retention members 210, 220 may be formed from a combination of materials (e.g., the material forming the underlying stent structure, as well as a coating or liner more flexible than the stent material, such as described above), whereas the intermediate section 230 may be simply formed of a flexible material more flexible than the stent material. In other words, even if formed of the same material forming the coating or liner of the retention members 210, 220, the intermediate section 230 would lack the additional support structure present in the retention members 210, 220. In some embodiments, the intermediate section 230 is formed of a substantially solid wall of flexible material (e.g., in contrast with a woven material which may have interstices therealong).

In embodiments in which an implantable medical device is configured and deployed to form an anastomosis between apposed anatomical structures, the intermediate section thereof may be in the form of a generally cylindrical tube with a lumen defined therethrough. For instance, in the example of an embodiment of an implantable medical device 200 illustrated in FIG. 2, a lumen 235 defined through the intermediate section 230, along with lumens 215, 225 defined respectively through the retention members 210, 220, together define a lumen 205 through the implantable medical device 200 forming an anastomosis between the apposed anatomical structures PS and DS. In accordance with various principles of the present disclosure, the intermediate section 230 can be of a material that is flexible while maintaining the lumen 235 through the intermediate section 230 in an open configuration. For instance, a reinforcement structure, such as a braided layer, circumferential or helical reinforcements, and/or ribbed or corrugated sections may be included within or along the wall of the intermediate section 230 to impart hoop strength, radial force, and/or other resistance to collapse and closure of the lumen 235 through the intermediate section 230. The retention members 210, 220 typically are configured to maintain the lumens 215, 225 therethrough in an open configuration, thereby maintaining, with the intermediate section 230, the open configuration of the lumen 205 through the implantable medical device 200.

An intermediate section 230 formed to be distinct from the retention members 210, 220 of an implantable medical device 200 formed in accordance with various principles of the present disclosure is nonetheless coupled with the retention members 210, 220. The intermediate section 230 may be secured to the retention members 210, 220 in a manner which holds the retention members 210, 220 in position with respect to the deployment site. Moreover, the intermediate section 230 should be secured to the retention members 210, 220 so as not detach from the retention members 210, 220 during the duration of use within a patient's body. The distal end 231 of the intermediate section 230 may thus be secured to the distal retention member 210, and the proximal end 233 of the intermediate section 230 may thus be secured to the proximal retention member 220 in any desired manner which maintains the connection thereto during the dwell time or the duration of use of (aka, “life” of) the implantable medical device 200. For instance, the intermediate section 230 may be formed separately from the retention members 210, 220 and then securely coupled thereto. More particularly, the intermediate section 230 may be formed by dipping, painting, spraying, or otherwise coating and/or covering a mandrel, such as a Teflon coated mandrel, with the polymeric material from which the wall of the intermediate section 230 is to be formed. The polymeric material typically is in a liquified form during formation of the intermediate section 230 (e.g., during application to a mandrel or mold or other element forming the ultimate shape of the finished intermediate section 230), and is allowed to cool, harden, solidify, cure, or otherwise form the wall of the finished intermediate section 230. The respective ends of the thus formed intermediate section 230 may be affixed (e.g., bonded, adhered, sutured, interwoven, attached, etc.) to the retention members 210, 220, such as by using suitable glues, adhesives, resins, or other bonding techniques. The material from which the intermediate section 230 is formed may be amenable to being bonded to the material or materials forming the retention members 210, 220. For instance, if one or both of the retention members 210, 220 has a coating or liner such as described above, the material of the intermediate section 230 may readily bond with the material of such coating or liner of the retention members 210, 220, particularly if formed of the same materials. The material forming the intermediate section 230 may be used to form a coating or liner provided with respect to the retention members 210, 220. Optionally, such material may be applied to the retention members 210, 220 during formation of the intermediate section 230, thereby coupling such sections of the implantable medical device 200. Various techniques available to those of ordinary skill in the art may be used to form an intermediate section 230 in accordance with various principles of the present disclosure, the present disclosure not being limited in this regard.

In some embodiments, the retention members 210, 220 are structurally configured to facilitate bonding of the ends 231, 233 of the intermediate section 230 to the retention members 210, 220. For instance, the retention members 210, 220 may include flanges or shoulders or other structures shaped to facilitate coupling or anchoring of the intermediate section 230 thereto. In the example of an embodiment of an implantable medical device 200 illustrated in FIG. 2, the retention members 210, 220 have respective extensions 216, 226 extending towards the intermediate section 230 of the implantable medical device 200. More particularly, the distal retention member 210 has an extension 216 extending from the tissue-engaging side 212t of the distal retention member 210 toward the proximal retention member 220. Similarly, the proximal retention member 220 has an extension 226 extending from the tissue-engaging side 222t of the proximal retention member 220 toward the distal retention member 210. The extensions 216, 226 are sized, shaped, configured, and/or dimensioned to facilitate coupling with the ends 231, 233 of the intermediate section 230. For instance, one or both of the extensions 216, 226 may be substantially cylindrical with a diameter selected so that the ends 231, 233 of the intermediate section 230 may either fit over or within the extensions 216, 226 and then be secured thereto. It will be appreciated that the bond between the intermediate section 230 and the retention members 210, 220 may be formed and/or have finished characteristics or properties selected in view of the manner in which the implantable medical device 200 is to be used (e.g., based on the deployment site, anticipated tensile or pullout forces to impact the implantable medical device 200, etc.). It is noted that the extensions 216, 226 may be structurally similar to optional outwardly-extending extensions 118, 128, 218, 228 of the retention members 110, 120, 210, 220 (extending in a direction away from the intermediate sections 130, 230) of the examples of embodiments of implantable medical devices 100, 200. Any or all such extensions 116, 118, 216, 218, 216, 228 may facilitate finishing of a woven material (e.g., providing more ready access to ends of the woven filaments to weld or otherwise treat the filaments so as not to unravel), manipulation (e.g., delivery and/or withdrawal) of the associated implantable medical device 100, 200, etc.

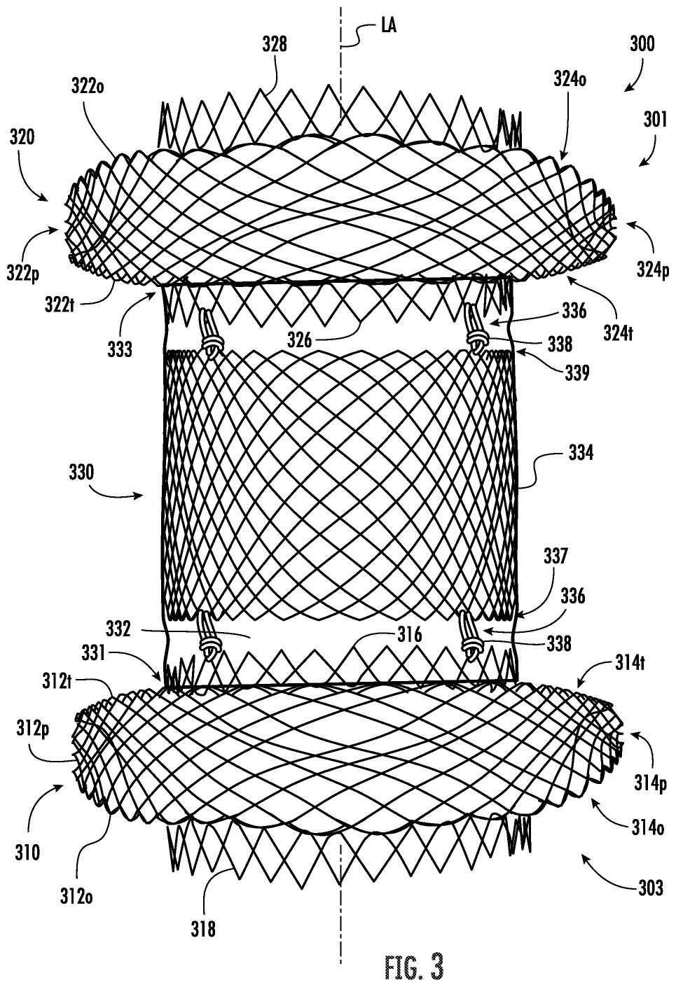

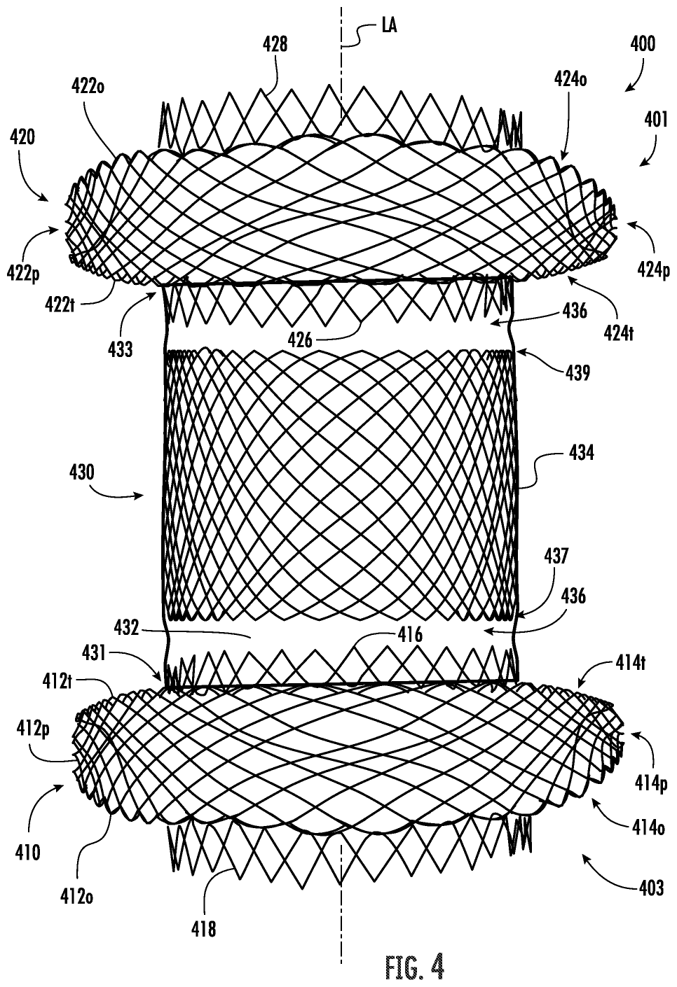

Alternatively, the intermediate section of an implantable medical device formed in accordance with various principles of the present disclosure may be formed of knitted or otherwise interwoven filaments, rather than a layer of generally solid material (e.g., a silicone wall). The weave pattern and/or the pitch of the weave or other structural characteristic of the intermediate section can be adjusted, as desired or necessary, to achieve an ascertained flexibility relative to the retention members. An example of an embodiment of an implantable medical device 300 with retention members 310, 320 as well as at least a portion of an intermediate section 330 formed of a woven or knitted material is illustrated in FIG. 3. It is noted that although the retention members 310, 320 are illustrated as double-walled retention members with respective first walls 314t, 324t and second walls 3140, 3240 adjoined by peripheral walls 314p, 324p, respectively defining distal tissue-engaging sides 312t, 322t, proximal tissue-engaging side 312t, 322t, and peripheral sides 312p, 322p, the retention members 310, 320 may be simply single-walled retention members, the present disclosure not being limited in this regard. Moreover, the outwardly-extending extensions 318, 328 are optional. The woven portion 334 of the intermediate section 330 of the implantable medical device 300 may have a flexibility (or resistance to flexing) similar to or different from that of the retention members 310, 320. In such embodiment, a dynamic section of the intermediate section 330 may be formed or provided or at least enhanced by flexible connecting regions 336 which couple the distal end 337 of the woven portion 334 of the intermediate section 330 with the distal retention member 310, and the proximal end 339 of the woven portion 334 of the intermediate section 330 with the proximal retention member 320. The connecting regions 336 are configured to have sufficient flexibility such that forces imparted to the intermediate section 330 are absorbed or dissipated or otherwise not transmitted to the retention members 310, 320. In the example of an embodiment of an implantable medical device 300 illustrated in FIG. 3, the connecting regions 336 are or include sutures 338. However, other configurations of connecting regions 336 are within the scope and spirit of the present disclosure. For instance, an example of an embodiment of an implantable medical device 400 similar to the implantable medical device 300 of FIG. 3 is illustrated in FIG. 4 without sutures (such as in the implantable medical device 300 of FIG. 3) coupling the woven portion 434 of the intermediate section 430 thereof with the respective retention members 410, 420 thereof. It will be appreciated that the other features and elements of the implantable medical device 400 illustrated in FIG. 4 which are similar to features and elements of the implantable medical device 300 illustrated in FIG. 3 are labeled with the same reference characters with the numerical portion thereof increased by 100, reference being made to the descriptions thereof with respect to the implantable medical device 300 for the sake of brevity and without intent to limit. In some embodiments, the distal and proximal ends 331, 333 of the intermediate section 330 are not directly coupled to the retention members 310, 320, thereby further distinguishing the properties or characteristics of these sections of the implantable medical device 300 from each other.

In the example of an embodiment of an implantable medical device 300 illustrated in FIG. 3, the intermediate section 330 includes a sleeve or liner 332, which may be similar to the intermediate section 230 of the implantable medical device 200 of FIG. 2, or similar to other liners of saddle regions of stents known to those of ordinary skill in the art. The liner 332 may extend the full length of the intermediate section 330, or may extend less than the full length of the intermediate section 330. The liner 332 may extend at least along the ends 331, 333 of the intermediate section 330. The liner 332 may extend across a gap between the ends 337, 339 of the woven portion 334 of the intermediate section 330, and the retention members 310, 320 of the implantable medical device 300, such as to couple the intermediate section 330 with the retention members 310, 320. Additionally or alternatively, the liner 332 may form, or at least partially form, the connecting regions 336 and/or one or more dynamic sections of the intermediate section 330 of the implantable medical device 300. In an embodiment in which the liner 332 forms one or more dynamic sections of the intermediate section 330 of the implantable medical device 300, the liner 332 has sufficient flexibility to allow the above-described flexing, pivoting, etc., of the intermediate section 330 without dislodging or otherwise affecting the retention members 310, 320. By extending across gaps or discontinuities between the ends 337, 339 of the woven portion 334 of the intermediate section 330, and the retention members 310, 320, the liner 332 may maintain axial flow through the implantable medical device 300 (and inhibit lateral flow through the walls of the implantable medical device 300). Similar to the intermediate section 230 of the example of an embodiment of an implantable medical device 200 illustrated in FIG. 2, the ends 331, 333 of the intermediate section 330 of the example of an embodiment of an implantable medical device 300 illustrated in FIG. 3 may be coupled to extensions 316, 326 similar to the above-described extensions 216, 226. For the sake of brevity, reference is made to the above description of the extension 216, 226 of the example of an embodiment illustrated in FIG. 2 as applicable to the extensions 316, 326 of the example of an embodiment illustrated in FIG. 3.

In view of the above, it should be understood that the various embodiments illustrated in the figures have several separate and independent features, which each, at least alone, has unique benefits which are desirable for, yet not critical to, the presently disclosed implantable medical device. Therefore, the various separate features described herein need not all be present in order to achieve at least some of the desired characteristics and/or benefits described herein. Only one of the various features may be present in an implantable medical device formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

Although embodiments of the present disclosure may be described herein with specific reference to medical devices and systems and procedures for treating the gastrointestinal system, it should be appreciated that such medical devices, systems, and methods may be used to treat anatomical structures of the abdominal cavity, digestive system, urinary tract, reproductive tract, respiratory system, cardiovascular system, circulatory system, etc. For instance, the anatomical structures with respect to which implantable medical devices of the present disclosure are deployed may be organs, cysts, pseudocysts, muscles, lumens, channels, vessels, passages, anatomical walls, etc., which are separate or separated apart from each other and/or independent of each other, whether different portions of the same anatomical structure, or different anatomical structures. It will further be appreciated that the present disclosure is not limited to use between particular anatomical structures and/or use to hold anat. For the sake of convenience, and without intent to limit, reference may be made to holding tissue walls in apposition, it being appreciated that such is only one example of anatomical structures and association therewith for which principles of the present disclosure are applicable. Moreover, for the sake of convenience, and without intent to limit, reference is made herein to non-adherent anatomical structures. Various further benefits of the various aspects, features, components, and structures of an implantable medical device such as described herein, in addition to those discussed above, may be appreciated by those of ordinary skill in the art.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable medical device for implanting with respect to first and second anatomical structures, the implantable medical device comprising:
a first end;
a second end;
an intermediate section defined between said first end and said second end; and
at least one dynamic section along at least a part of said intermediate section;
wherein:
said first end and said second end are configured to hold the first and second anatomical structures in apposition with said intermediate section extending through the first and second anatomical structures;
said first end of said implantable medical device is configured to be anchored with respect to the first anatomical structure;
said second end of said implantable medical device is configured to be anchored with respect to the second anatomical structure;
said intermediate section is configured to hold the first and second anatomical structures in apposition, with said first end of the implantable medical device anchored with respect to the first anatomical structure and said second end of said implantable medical device anchored with respect to the second anatomical structure; and
said at least one dynamic section is configured to allow movement of said first end of said implantable medical device relative to said second end of said implantable medical device.

2. The implantable medical device of claim 1, wherein the dynamic section is substantially coextensive with the intermediate section.

3. The implantable medical device of claim 1, wherein:
at least said first end and said second end of said implantable medical device are formed of interconnected filaments; and
said at least one dynamic section comprises a structure different from the interconnected filament structure of said first end and said second end.

4. The implantable medical device of claim 3, wherein said at least one dynamic section is formed of a substantially solid polymeric wall.

5. The implantable medical device of claim 4, wherein said at least one dynamic section is substantially coextensive with portions of said intermediate section not comprising said at least one dynamic section.

6. The implantable medical device of claim 3, wherein:
said intermediate section includes a section formed of interconnected filaments; and
said at least one dynamic section includes a first dynamic section coupling a first end of said interconnected filament section of said intermediate section to said first end of said implantable medical device, and a second dynamic section coupling a second end of said interconnected filament section of said intermediate section to said second end of said implantable medical device.

7. The implantable medical device of claim 1, wherein:
a first retention member extends radially-outwardly along said first end of said implantable medical device;
a second retention member extends radially-outwardly along said second end of said implantable medical device;
said first retention member and said second retention member are formed from filaments interconnected in a first pattern; and
at least a portion of said intermediate section is formed from filaments interconnected in a second pattern different from the first pattern.

8. The implantable medical device of claim 7, wherein the second pattern imparts a different degree of flexibility to said intermediate section than the flexibility imparted by the first pattern to said first retention member and to said second retention member.

9. The implantable medical device of claim 8, wherein said at least one dynamic section comprises a first dynamic section coupling a first end of said portion of said intermediate section formed from interconnected filaments with said first retention member, and a second dynamic section coupling a second end of said portion of said intermediate section formed from interconnected filaments with said second retention member.

10. The implantable medical device of claim 1, wherein a lumen is defined through said implantable medical device to form an anastomosis between the apposed anatomical structures.

11. The implantable medical device of claim 10, wherein said at least one dynamic section is reinforced to maintain the lumen through said implantable medical device in an open configuration.

12. The implantable medical device of claim 1, wherein said intermediate section includes more than one dynamic section.

13. The implantable medical device of claim 1, wherein:
said implantable medical device is shiftable from a delivery configuration to an expanded deployed configuration;
said first end of said implantable medical device expands when said implantable medical device shifts to said deployed configuration to define a radially-outwardly extending first retention member;
said second end of said implantable medical device expands when said implantable medical device shifts to the deployed configuration to define a radially-outwardly extending second retention member;
a first end of said intermediate section is coupled to said first retention member; and
a second end of said intermediate section is coupled to said second retention member.

14. The implantable medical device of claim 1, wherein said intermediate section is sized, shaped, configured, and/or dimensioned to hold adjacent non-adherent anatomical structures in apposition.

15. An implantable medical device configured to hold first and second anatomical structures in apposition, said implantable medical device having a first end and a second end with an intermediate section extending therebetween, said implantable medical device comprising:
a first retention member sized, shaped, configured, and/or dimensioned to anchor a first end of said implantable medical device with respect to the first anatomical structure;
a second retention member sized, shaped, configured, and/or dimensioned to anchor a second end of said implantable medical device with respect to the first anatomical structure; and
a dynamic section defined along said intermediate section to allow said intermediate section to be movable with respect to said first retention member and said second retention member.

16. The implantable medical device of claim 15, wherein:
said first retention member and said second retention member are formed from interconnected filaments; and
said dynamic section comprises a structure different from the interconnected filament structure of said first retention member and said second retention member.

17. A method of holding a first anatomical structure of a patient in apposition with a second anatomical structure of the patient, said method comprising;
anchoring a first end of an implantable medical device with respect to the first anatomical structure;
extending an intermediate section of the implantable medical device across the first anatomical structure and the second anatomical structure;
anchoring a second end of the implantable medical device with respect to the second anatomical structure; and
allowing a dynamic section of the implantable medical device to flex with respect to at least one of the retention members;
wherein:
the intermediate section is sized, shaped, configured, and/or dimensioned to hold the first and second anatomical structures in apposition with the first end of the implantable medical device anchored with respect to the first anatomical structure and the second end of the implantable medical device anchored with respect to the second anatomical structure.

18. The method of claim 17, wherein a lumen is defined through the implantable medical device, said method further comprising forming an anastomosis between the first and second anatomical structures.

19. The method of claim 18, wherein the first anatomical structure is the jejunum of the patient and the second anatomical structure is the stomach of the patient, said method further comprising forming a gastrojejunostomy.

20. The method of claim 19, further comprising:
delivering the implantable medical device, in a delivery configuration, transluminally through the patient;
deploying the first end of the implantable medical device in the first anatomical structure and allowing the first end of the implantable medical device to expand to form a first retention member anchored against a distal side of the first anatomical structure;
extending the intermediate section through a wall of the first anatomical structure and a wall of a second anatomical structure; and
deploying the second end of the implantable medical device in the second anatomical structure and allowing the second end of the implantable medical device to expand to form a second retention member anchored against a proximal side of the second anatomical structure.
